(19) United States
(12) Patent Application Publication
(10) Pub. No.: US 2011/0091933 A1
(43) Pub. Date: Apr. 21, 2011

(54) METHOD OF TREATING MICROORGANISM

(75) Inventor: Hideyuki KANEHARA, Kanagawa (JP)

(73) Assignee: FUJIFILM CORPORATION, Tokyo (JP)

(21) Appl. No.: 12/904,397

(22) Filed: Oct. 14, 2010

(30) Foreign Application Priority Data
Oct. 20, 2009 (JP) 2009-241663

(51) Int. Cl. C12Q 1/06; C12N 1/00; C12N 1/12

(52) U.S. Cl. 435/39; 435/243; 435/257.1

(57) ABSTRACT

A method of treating microorganisms which can solve conventional problems in a treatment of counting the number of microorganisms, a proliferation treatment of microorganisms and a purification treatment of microorganisms is provided. A method of treating microorganisms in which any one of the intended treatments of a treatment of counting the number of microorganisms, a proliferation treatment of microorganisms and a purification treatment of microorganisms is carried out, the method including, before carrying out an intended treatment step, a pretreatment step of packing a predetermined amount of a sample solution containing the microorganisms in a closed container 22 and subjecting the closed container to high speed oscillating motion.

### BACKGROUND OF THE INVENTION

### 1. Field of the Invention

The present invention relates to a method of treating microorganisms, and particularly to a pretreatment technique before carrying out any one of the intended treatments of a treatment of counting the number of microorganisms, a proliferation treatment of microorganisms, and a purification treatment of microorganisms.

2. Description of the Related Art

Treatments of microorganisms include treatments of counting the number of microorganisms, proliferation treatments of microorganisms, and purification treatments of microorganisms, and so far these have been carried out as described below.

### <Treatment of Counting the Number of Microorganisms>

When counting the number of microorganisms, generally cytometers have been used to observe a sample solution containing microorganisms (Sorui Kenkyuho “Approach to Study Algae” edited by Kazutoshi Nishizawa and Mitsuo Chihara, KYORITSU SHUPPAN CO., LTD, 1979, p275). This method does not cause any problem when each of the microorganisms is separated, but when the microorganisms are aggregated, often it is not easy to count the number because they overlap one another. Further, in the case of sample solutions composed of several types of aggregated microorganisms, it has even been impossible to count the number of microorganisms per species.

Thus, a method of disintegrating aggregates of microorganisms by an ultrasonic treatment on a sample solution has been adopted (e.g., Japanese Patent Application Laid-Open No. 7-298869). However, as can be expected from the ultrasonic treatment being used for cleaving nucleic acid, the method has the problem that the cell wall of the microorganisms is damaged and thus the microorganisms are killed depending on the ultrasonic treatment conditions. Further, different microorganisms have different hardness of cell walls, and so it is very complicated and inefficient for workers to determine ultrasonic treatment conditions that avoid damage to cell walls for each type of microorganisms.

As an alternative method of counting the number of microorganisms, the concentration of microorganisms can be measured based on the turbidity of the microorganisms, but the relationship between the number of the microorganisms and the turbidity needs to be determined in advance. Therefore, it is also necessary to count the number of microorganisms even in this method, and thus the only way has been to count aggregated microorganisms patiently. Moreover, when aggregated microorganisms are counted based on the turbidity, irregular reflection of light is likely to be caused, and so a special instrument such as an integrating sphere has been needed.

Also, in the case of photosynthetic microorganisms containing chlorophyll in particular, the number of the microorganisms has been counted based on the quantitative determination of chlorophyll. However, it is necessary to determine the correlation between the chlorophyll quantity and the number of microorganisms in advance even in this method, and the method has the same difficulty as described above. Moreover, since the chlorophyll quantity in microorganisms varies depending on the culturing conditions or proliferation stages of microorganisms, the correlation between the chlorophyll quantity and the number of microorganisms must be determined for each case, making the procedure complicated.

Still another method of counting the number of microorganisms is to use flow cytometry. This equipment, however, is difficult to be used for aggregates of microorganisms because a microorganism must be passed through a narrow tube having a diameter almost the same as the size of the microorganism and because when an aggregate of some microorganisms flow through the tube, the number of the microorganisms is recognized to be one.

### <Proliferation Treatment of Microorganism>

Prokaryotes such as Escherichia coli divide in tens of minutes and thus have a very high proliferation rate. On the other hand, eukaryotes, for example, algae require a considerable time for a division. For example, Botryococcus Braunii, a type of algae which accumulate oil, which has attracted attention as a biomass fuel and is regarded as an important microorganism to curb global warming, requires a few days to a few weeks for a division, and is known to have an extremely low proliferation rate. This is one of the causes of the current condition that oil production using Botryococcus Braunii has not been done on a commercial scale.

So far it has been studied to design culturing conditions of microorganisms to improve the proliferation rate, and various methods of improving the proliferation rate have been reported. However, the proliferation rate is unsatisfactory at the present.

### <Purification Treatment of Microorganism>

Conventional methods of purifying microorganisms involve diluting a solution containing microorganisms that has been collected and applying it on an agar medium. Generally, however, it is rare that a single microbe is obtained through one purification step, and the purification step is usually repeated a few times to obtain a single microbe. In the case of microorganisms forming aggregates or microorganisms in a biofilm which are generally composed of a plurality of types of microorganisms, they are subjected to purification with a few types of microorganisms being mixed when merely diluted. This causes a large increase in the number of repeat of purification steps and consequently makes the purification very difficult.

### SUMMARY OF THE INVENTION

As described above, conventional treatments of counting the number of microorganisms, proliferation treatments of microorganisms and purification treatments of microorganisms each have problems, and neither of them are satisfactory.

The present invention has been made under such circumstances and an object of the present invention is to provide a method of treating microorganisms which can solve conventional problems in a treatment of counting the number of microorganisms, a proliferation treatment of microorganisms, and a purification treatment of microorganisms.

Accordingly, for the treatment of counting the number of microorganisms, even if aggregates of microorganisms are present in a sample solution, the aggregates can be efficiently disaggregated while preventing disruption of microorganisms, and thus a treating method of easily and accurately counting the number of microorganisms can be provided.

Also, for the proliferation treatment of microorganisms, even if aggregates of microorganisms are present in a sample solution, the aggregates can be efficiently disaggregated while preventing disruption of microorganisms, and thus a treating method capable of significantly improving proliferation rates compared to conventional methods even for microorganisms having a low proliferation rate can be provided.

Also, for the purification treatment of microorganisms, even if aggregates of microorganisms are present in a sample solution, the aggregates can be efficiently disaggregated while preventing disruption of microorganisms, and thus a treating method capable of significantly reducing the number of repeat of purification steps and significantly reducing the time of purification compared to conventional methods is provided.

To achieve the aforementioned object, a first aspect of the present invention provides a method of treating microorganisms in which any one of the intended treatments of a treatment of counting the number of microorganisms, a proliferation treatment of microorganisms and a purification treatment of microorganisms is carried out, the method including, before carrying out the intended treatment step, a pretreatment step of packing a predetermined amount of a sample solution containing the microorganisms in a closed container and subjecting the closed container to high speed oscillating motion.

According to the method of treating microorganisms of the present invention, a predetermined amount of a sample solution containing microorganisms is packed in a closed container and the closed container is subjected to high speed oscillating motion as a pretreatment step for the treatment of counting the number of microorganisms, proliferation treatment of microorganisms and purification treatment of microorganisms.

In the present invention, lumps of aggregates of microorganisms are disaggregated in a sample solution by subjecting a closed container to high speed oscillating motion, not by subjecting microorganisms to direct vibration as in conventional ultrasonic vibration. As a result, aggregates can be effectively disaggregated while preventing disruption of microorganisms.

In consideration of the disaggregation efficiency for such aggregates, the sample solution is packed in a closed container in a predetermined amount of preferably 15 to 75% by volume in terms of the volume ratio of the sample solution to the inner volume of the closed container.

This is because when a sample solution containing microorganisms is packed in a closed container without creating a given volume of a head space in the closed container, the disaggregation efficiency for aggregates of microorganisms (hereinafter sometimes simply referred to as aggregates) is remarkably reduced when the closed container is subjected to high speed oscillating motion. This is also because if the amount of the sample solution in the closed container is too small, the disaggregation efficiency for aggregates of microorganisms is remarkably reduced upon the high speed oscillating motion. The reason seems to be because the weight of the sample solution is too low and so the collision force against the inner wall of the closed container upon high speed oscillating motion is small, and therefore energy needed to disperse aggregates cannot be fully achieved.

Also, for the high speed oscillating motion which enables efficient dispersion of aggregates while preventing disruption of microorganisms, it is preferred that the closed container is oscillated so that a value of oscillating number (rpm)×oscillating time (second) ranges 10,000 to 3,000,000. This is because the above combination of the oscillating number and the oscillating time enables efficient dispersion of aggregates of a wide variety of microorganisms while preventing disruption of microorganisms.

Thus, in the treatment of counting the number of microorganisms, even if aggregates of microorganisms are present in a sample solution, the aggregates can be efficiently disaggregated while preventing disruption of microorganisms, and therefore the number of microorganisms can be counted easily and accurately.

Also, in the proliferation treatment of microorganisms, aggregates can be efficiently disaggregated while preventing disruption of microorganisms, and thus individual microorganisms have an easy access to components necessary for proliferation, such as nutrient sources, and can take in the components sufficiently. As a result, proliferation rates can be significantly improved compared to those in conventional methods even for microorganisms having a low proliferation rate.

Also, in the purification treatment of microorganisms, aggregates can be efficiently disaggregated while preventing disruption of microorganisms, and thus it becomes easier to extract microorganisms to be purified in a state that the microorganisms are distinguished from saprophytes. As a result, the number of repeat of purification steps can be significantly reduced compared to those in conventional methods, and therefore the time of purification (the number of steps) can be significantly reduced.

In these cases, when the disaggregation effect of aggregates falls short of the desired level, beads such as resin particles may be packed in the closed container to the extent that the disruption of microorganisms can be prevented. Basically, however, it is preferred that the disaggregation effect is adjusted by combining the oscillating number and the oscillating time without packing beads.

In the method of treating microorganisms of the present invention, the oscillating pattern for the closed container is preferably at least one pattern in a vertical direction, a horizontal direction or a back and forth direction. Here, high speed oscillating motion in which at least two patterns in vertical, horizontal, or back and forth directions are combined is even more preferred.

In the pretreatment step of the method of treating microorganisms of the present invention, an antifoaming agent is preferably added to the closed container in which a sample solution is packed.

This is because when a sample solution contains microorganisms at a high concentration or microorganisms excrete various metabolites to the outside of their bodies, bubbles are generated in the closed container in the pretreatment step, and the bubbles decrease the disaggregation efficiency for aggregates of microorganisms.

In the method of treating microorganisms of the present invention, the above pretreatment step is preferably carried out in several stages. This is to avoid undesirable results such as death of microorganisms due to the temperature increase of the sample solution caused by the high speed oscillating motion in the pretreatment step. This is particularly important for the pretreatment step for microorganisms vulnerable to heat.

In the method of treating microorganisms of the present invention, it is preferred that the microorganisms have aggregation properties.

This is because the present invention is even more effective for treating microorganisms having aggregation properties. Examples of microorganisms having aggregation properties include adherent microorganisms.

In the method of treating microorganisms of the present invention, a complex including a plurality of types of microorganisms may be used as the aforementioned microorganisms.

In the method of treating microorganisms of the present invention, it is preferred that the microorganisms produce biomass material.

This is because the method of treating microorganisms of the present invention is very useful as a technology for the industrialization of biomass fuel or the like using microorganisms which produce biomass material, such as microorganisms of the genus Botryococcus containing oil.

In the present invention, “biomass material” includes materials used for fuel, medical product, cosmetic product, and the like.

In the method of treating microorganisms of the present invention, it is preferred that the pretreatment step and the intended treatment step are repeated several times when the intended treatment step is the proliferation treatment of microorganisms.

This is because since microorganisms are likely to be aggregated again in the proliferation treatment step, the proliferation rate can be further improved by repeating the pretreatment step and the proliferation treatment step.

According to the method of treating microorganisms of the present invention, the conventional problems in the treatment of counting the number of microorganisms, the proliferation treatment of microorganisms and the purification treatment of microorganisms can be solved.

Specifically, in the treatment of counting the number of microorganisms, even if aggregates of microorganisms are present in a sample solution, the aggregates can be efficiently disaggregated while preventing disruption of microorganisms, and therefore the number of microorganisms can be counted easily and accurately.

Also, in the proliferation treatment of microorganisms, even if aggregates of microorganisms are present in a sample solution, the aggregates can be efficiently disaggregated while preventing disruption of microorganisms, and therefore proliferation rates can be significantly improved compared to those in conventional methods even for microorganisms having a low proliferation rate.

Also, in the purification treatment of microorganisms, even if aggregates of microorganisms are present in a sample solution, the aggregates can be efficiently disaggregated while preventing disruption of microorganisms, and therefore the number of repeat of purification steps can be significantly reduced compared to those in conventional methods, and the time of purification (the number of steps) can be significantly reduced.

### BRIEF DESCRIPTION OF THE DRAWINGS

FIG. 1 is an exterior view illustrating an example of a pretreatment apparatus used in the method of treating microorganisms of the present invention;

FIG. 2 is a perspective view illustrating a container holder and a drive unit of the pretreatment apparatus shown in FIG. 1;

FIG. 3 is a perspective view illustrating how to pack microorganisms in a closed container used in the pretreatment apparatus;

FIGS. 4A to 4C are explanatory views illustrating various oscillating patterns for a closed container in the pretreatment step;

FIG. 5 is a micrograph of microalgae before the pretreatment step in Example A;

FIG. 6 is a micrograph of microalgae after the pretreatment step in Example A;

FIG. 7 is a micrograph of microalgae after conventional ultrasonic irradiation in Example A;

FIG. 8 is a micrograph illustrating the disruption of cells of microalgae after conventional ultrasonic irradiation in Example A;

FIG. 9 is a micrograph illustrating the disruption of cells of microalgae after high speed oscillating motion with beads added to a closed container together with microorganisms in Example A;

FIG. 10 is an explanatory view illustrating the relationship between the volume ratio of a sample solution to the inner volume of a closed container and the disaggregation effect of aggregates of microorganisms in Example B;

FIG. 11 is a micrograph of Botryococcus before the pretreatment step in Example C;

FIG. 12 is an explanatory view illustrating the relationship between the vibration for a closed container in the pretreatment step and the disaggregation effect in Example C;

FIG. 13 is an explanatory view illustrating the relationship between the value of oscillating number×oscillating time for a closed container in the pretreatment step and the disaggregation effect in Example C;

FIG. 14 is an explanatory view illustrating the impact of the presence or absence of the pretreatment step on the proliferation of microalgae in Example D; and

FIG. 15 is an explanatory view illustrating the relationship between the value of oscillating number×oscillating time for a closed container in the pretreatment step and the proliferation effect of microalgae in Example E.

### DETAILED DESCRIPTION OF THE PREFERRED EMBODIMENTS

Hereinafter preferred embodiments of the method of treating microorganisms of the present invention will be described in detail.

The present invention includes an intended treatment step of carrying out any one of the intended treatments of a treatment of counting the number of microorganisms, a proliferation treatment of microorganisms, and a purification treatment of microorganisms, and a pretreatment step of packing a predetermined amount of a sample solution containing microorganisms in a closed container and subjecting the closed container to high speed oscillating motion before carrying out the intended treatment step.

### [Microorganisms]

Microorganisms according to the present invention refer to microscopic organisms whose individuals are indiscernible to the human eye. Both of prokaryotes and eukaryotes may be used. The prokaryotes are organisms that lack a cell nucleus and include two organisms of bacteria and archaea. The eukaryotes include algae, protists, fungi, myxomycetes and also microscopic animals such as rotifers.

Further, some microorganisms are aggregated with each other and thus are discernible to the human eye. Even in such cases, when the complex structure is separated into individual microorganisms and the individual microorganisms are indiscernible to the human eye, such microorganisms are defined as the microorganism in the present invention. There are also microorganisms which are in an individually separated state and whose presence can be recognized by the change of colors caused by an increase in the number of the microbial bodies (e.g., the number of microalgal bodies). Even in such cases, however, each microorganism cannot be observed by the human eye, and therefore they are included in the microorganism according to the present invention.

Algae according to the present invention mean oxygen-evolving photosynthetic organisms excluding bryophytes, pteridophytes and seed plants which live on ground, and are a general name for different groups including cyanobacteria which are a prokaryote and eukaryotes such as unicellular or multicellular seaweed.

The microalgae in the present invention mean a group of organisms which is the above-described algae defined in the present invention selected from the microorganisms defined in the present invention. The microalgae refer to, for example, species of Cyanophyta, Glaucophyta, Rhodophyta, Chlorophyta, Cryptophyta, Haptophyta, Heterokontophyta, Dinophyta, Euglenophyta and Chlorarachniophyta.

Microorganisms which produce biomass material are more preferred as the microorganism used in the present invention. Herein, the biomass means resources derived from living organisms, renewable organic resources excluding fossil resources; such as organism-derived substances, food, materials, fuel and resources. The biomass material includes polysaccharide and oil (such as hydrocarbon compounds and triglyceride) produced by organisms. Specific examples of biomass material-producing microorganisms include oil-containing microorganisms of the genus Botryococcus. The present invention is particularly useful as a technology for the industrialization of biomass fuel or the like using microorganisms of the genus Botryococcus.

### [Pretreatment Step]

The pretreatment step according to the present invention is a step of packing a sample solution containing microorganisms in a closed container and subjecting the closed container to high speed oscillating motion, thereby disintegrating aggregates of the microorganisms to be disaggregated into individual (single) microorganisms while preventing disruption of the microorganisms even if the sample solution contains aggregates.

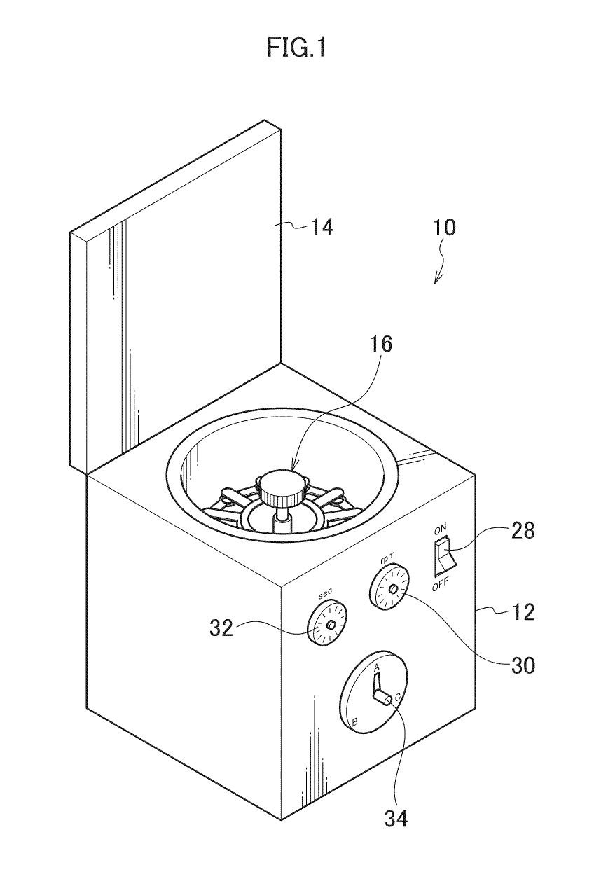

FIG. 1 to FIG. 3 illustrate an example of a pretreatment apparatus for carrying out the pretreatment step.

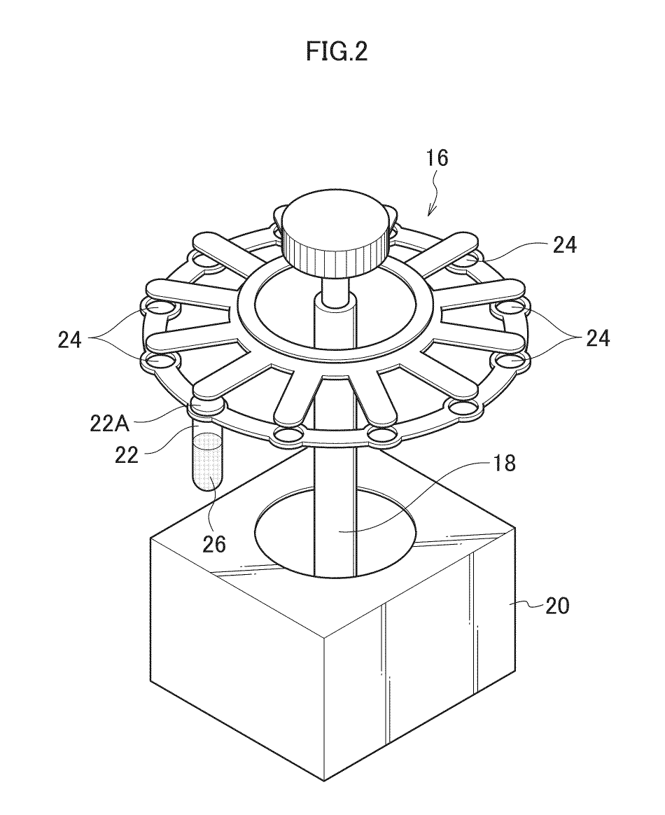

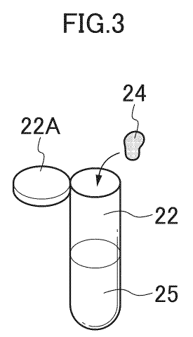

As FIG. 1 shows, the pretreatment apparatus 10 is constituted by an apparatus body 12 and a lid member 14. As FIG. 2 shows, a disk-shaped closed container holder 16 for high speed oscillating motion is arranged in the apparatus body 12 and supported by an oscillating drive unit 20 via a drive shaft 18. A plurality of chuck parts 24 which detachably hold a closed container 22 with a lid 22A (see FIG. 3) are provided on the periphery of the closed container holder 16.

As FIG. 3 shows, the closed container 22 with a lid 22A has a shape of a test tube and is structured so that the container can be sealed by the lid 22A after packing a sample solution 26 composed of microorganisms 24 and a dispersion 25 (for example, pure water or a culture solution) for dispersing the microorganisms.

Also, as FIG. 1 shows, an ON-OFF switch 28 and an oscillating number dial 30 for determining the oscillating number and a timer dial 32 for determining the oscillating time when the closed container 22 is subjected to high speed oscillating motion via the closed container holder 16 are provided at the front of the apparatus body 12. Further, a changeover switch 34 for determining the pattern of oscillating directions for subjecting the closed container 22 to high speed oscillating motion, or the like, is provided.

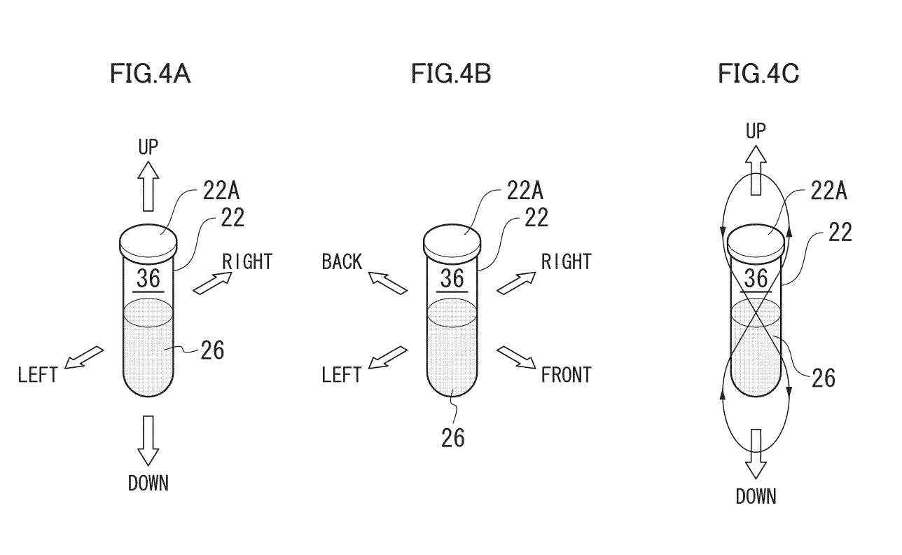

As FIGS. 4A to 4C show, the oscillating pattern for subjecting the closed container 22 to high speed oscillating motion is at least one pattern in a vertical direction, a horizontal direction or a back and forth direction. A combination of the vertical direction and the horizontal direction as shown in FIG. 4A or a combination of the horizontal direction and the back and forth direction as shown in FIG. 4B is preferably adopted. Moreover, a special oscillating pattern in which the closed container 22 is shaken in a figure of eight with vertically moving is even more preferably adopted.

As described above, in the pretreatment step according to the present invention, lumps of aggregates of microorganisms in the closed container 22 are disaggregated in a sample solution by subjecting the closed container 22 containing microorganisms to high speed oscillating motion, not by directly shaking the microorganisms themselves by ultrasonic vibration as in conventional methods. This enables efficient dispersion of aggregates of microorganisms while preventing disruption of microorganisms.

The disruption of microorganisms in the present invention means a phenomenon in which the outer membrane of microorganisms is broken and substances in the microorganisms flow out of the microorganisms. The occurrence of such disruption makes it impossible to accurately count the number of microorganisms in the treatment of counting the number of microorganisms described later. Moreover, the disruption causes disadvantages such as adversely affecting the cultivation of microorganisms.

However, it is difficult to prevent disruption of microorganisms completely even in the pretreatment step according to the present invention. “Preventing disruption of microorganisms” in the present invention means that 70% or more of the microorganisms are not disrupted. The level of the prevention of disruption is more preferably 80% or more, and particularly preferably 90% or more.

Also, by adding a dispersion to the closed container, not only the disaggregation efficiency for microorganisms is improved, but also the disruption of microorganisms can be reliably prevented compared to cases where the closed container 22 is subjected to high speed oscillating motion without adding a dispersion. Further, the addition of a dispersion to the closed container 22 can prevent deactivation or death of microorganisms due to temperature increase. The deactivation or death of microorganisms makes it impossible to count the number of cells in the intended treatment steps described later or has an adverse effect on the subsequent proliferation treatment.

Preferred conditions of the high speed oscillating motion for the closed container 22 for preventing the disruption of microorganisms and improving the disaggregation efficiency in the pretreatment step are determined by the combination of the oscillating number and the oscillating time. For the relationship between the oscillating number and the oscillating time, when the oscillating number is slightly high (for example, 5,000 rpm), sufficient disaggregation efficiency can be achieved in a short oscillating time (for example, 5 seconds). The value of oscillating number (rpm)×oscillating time (second) is 25,000 in that case. When the oscillating number is slightly low (for example, 3,500 rpm), the oscillating time is preferably longer (for example, 20 seconds). The oscillating number (rpm)×oscillating time (second) is 70,000 in that case.

On the other hand, when the oscillating number is too high (for example, higher than 10,000 rpm), a greater amount of heat is generated in the pretreatment apparatus 10. An increase in temperature caused by the heat generation is undesirable for the activity of common microorganisms excluding heat resistant bacteria. Also, the oscillating time is preferably 5 minutes (300 seconds) or less, more preferably 1 minute (60 seconds) or less in consideration of the disruption of microorganisms. The value of oscillating number (rpm)×oscillating time (second) at an oscillating number of 10,000 rpm for an oscillating time of 300 seconds is 3,000,000.

From the above, for the high speed oscillating motion, it is preferred that the closed container 22 is shaken so that the value of oscillating number (rpm)×oscillating time (second) ranges from 10,000 to 3,000,000. The value of oscillating number (rpm)×oscillating time (second) more preferably ranges from 70,000 to 330,000 based on the results of Examples described later. This is because the above combination of the oscillating number and the oscillating time enables efficient dispersion of aggregates of a wide variety of microorganisms while preventing disruption of such microorganisms.

Also, it is preferred that the sample solution 26 is packed in the closed container 22 in a volume ratio of the sample solution 26 to the inner volume of the closed container 22 of 15 to 75% by volume to create a head space 36 above the sample solution 26 (see FIG. 4). This is because when the closed container 22 is filled up with the sample solution 26, the disaggregation efficiency for aggregates is significantly reduced in the pretreatment with high speed oscillating motion. The volume ratio more preferably ranges from 20 to 60% by volume. The disaggregation efficiency is also significantly reduced when the volume ratio is too small, and thus the lower limit of the volume ratio is preferably 15% by volume.

When using a commercially available apparatus as a substitute for the high speed oscillating motion in the above pretreatment apparatus 10, Beads Cell Disrupter MS-100 made by TOMY SEIKO CO., LTD. (Tokyo) or Bead Beater-type Homogenizer BSP-3110BX made by BioSpec Products Inc. may be used.

However, these instruments are originally intended to disrupt microorganisms to extract nucleic acid from the microorganisms and disrupt cells by allowing microorganisms to collide with beads packed together with the microorganisms. Therefore, when such an instrument is directly used, the pretreatment step according to the present invention in which aggregates are efficiently disaggregated while preventing disruption of microorganisms cannot be achieved. In such a case, it is necessary to determine the particle size and the addition amount of beads to be packed in the closed container 22, conditions of high speed oscillating motion (oscillating number, oscillating time), and the level of disruption of microorganisms by a preliminary experiment or the like. For that reason, the apparatus is used without packing beads in the closed container 22 in the pretreatment step according to the present invention.

When the aggregation force of aggregates of microorganisms is strong and the disaggregation effect cannot be fully achieved, beads such as resin particles may be packed in the closed container 22 to the extent that the disruption of microorganisms can be prevented. Basically, however, it is preferred that the disaggregation effect is adjusted by combining the oscillating number and the oscillating time without packing beads as described above.

Also, when carrying out the pretreatment step, it is preferred that the pretreatment step is carried out not once but several times. This is to prevent the temperature increase of the sample solution 26 in the closed container 22 by dividing the pretreatment step into several parts. The temperature increase causes an undesirable effect such as death of microorganisms.

In the present invention, an antifoaming agent is preferably added to the solution of microorganisms when carrying out the pretreatment step. When the sample solution 26 contains microorganisms at a high concentration or microorganisms excrete various metabolites to the outside of their bodies, bubbles are generated in the closed container 22 in some cases in the pretreatment step. The generation of bubbles in the closed container 22 causes a remarkable decrease in the disaggregation efficiency for aggregates of microorganisms. In such cases, it is preferred that the pretreatment step is carried out after adding an antifoaming agent to the sample solution 26.

When the pretreatment step is employed as a pretreatment for a treatment of counting microorganisms, a proliferation treatment, and a purification treatment, it is preferred that the microorganisms disaggregated in the pretreatment step are immediately used. This is because the microorganisms may be reaggregated. If reaggregated, the microorganisms can be redisaggregated by using the pretreatment apparatus 10 described above.

### [Intended Treatment Step (Treatment of Counting the Number of Microorganisms)]

The treatment of counting the number of microorganisms means to count the number of microorganisms. The method of counting is not particularly limited. The number of microorganisms in the sample solution 26 obtained in the pretreatment step may be counted on a hemocytometer using a microscope, or may be automatically counted using a flow cytometer. Also, when microorganisms are directly counted using a light microscope, the number can be more clearly counted by staining microorganisms. Further, a method using a polarizing microscope or a differential interference contrast microscope instead of a bright field light microscope may be employed. In short, any method of observing appearances of microorganisms may be employed for the sample solution 26 which has been subjected to the pretreatment step of the present invention.

Alternatively, by determining the correlation between the absorbance and the number of microorganisms in advance, the number of microorganisms can be calculated from the absorbance by using an absorption spectrophotometer. Also, by determining the correlation between the turbidity and the number of cells in advance, the number of cells can be calculated from the turbidity by using a turbidimeter. Further, by determining the correlation between the fluorescence amount of chlorophyll and the number of cells in advance by a fluorophotometer, the number of cells can be calculated from the fluorescence amount of chlorophyll.

In any of the cases of counting microorganisms using the absorbance, turbidity or fluorescence amount of chlorophyll, it is necessary to determine the correlation with microorganisms in advance by a preliminary experiment. The pretreatment step according to the present invention can be suitably used for the preliminary experiment.

Also, by carrying out the pretreatment step according to the present invention, it becomes possible to use an image recognition software for counting the number of microorganisms. When such an image recognition software is used for aggregated microorganisms, the accuracy of recognition for individual microorganisms is much lower than that of the human eye. Therefore, the actual situation is that image recognition software could not have been used.

However, since the pretreatment step of the present invention allows aggregated microorganisms to be separated into individual microorganisms, maximum use of the recognition ability of image recognition software can be made. Furthermore, since the number of sells is not manually counted by a man but can be automatically counted, the workload of experimenters can be significantly reduced, and at the same time information such as the distribution of the size of cells can be obtained.

The aggregation in the present invention means a structure in which a plurality of microorganisms are assembled. The structure of microorganisms may be composed of some types of microorganisms or a single type of microorganisms. Further, microorganisms may be in direct contact or aggregated via a certain substance such as extracellular matrix. Those called a colony are also defined as the aggregation in the present invention.

As described above, while it was very difficult to count the number of aggregated microorganisms in conventional methods of counting the number of microorganisms, use of the present invention has made it easier to count the number of microorganisms because it allows aggregated microorganisms to be separated or makes aggregates smaller. For example, for Botryococcus Braunii which is known to accumulate oil inside and/or outside the algal bodies and to form aggregated colonies, a method utilizing absorption by chlorophyll or gravimetric measurement has been employed. In the present invention, however, the number can be easily counted only by carrying out the pretreatment step. Also, when microorganisms are ultrasonically disaggregated as in conventional methods of dispersing aggregates, structures such as nucleic acid in microorganisms are affected, causing adverse effects such as disruption of microorganisms. However, this will not happen in the pretreatment step according to the present invention.

Therefore, by carrying out the pretreatment step before the treatment of counting the number of microorganisms, the number of microorganisms can be counted easily and accurately.

### [Intended Treatment Step (Proliferation Treatment of Microorganisms)]

The proliferation treatment of microorganisms according to the present invention relates to the number of microorganisms present in a culture solution per unit volume of the culture solution after elapse of a certain time when microorganisms are cultured. Proliferation rates can be calculated based on the time elapsing from the start of cultivation to a certain point. In some cases the advantage of the pretreatment step according to the present invention can be restated in an expression of the improvement of the proliferation rate.

Also, when microorganisms are allowed to proliferate using microorganisms which have been subjected to the pretreatment step according to the present invention, aggregates may be formed again as the proliferation of the disaggregated microorganisms proceeds. In such a case, it is preferred that aggregates are treated in the pretreatment step again and allowed to proliferate again. The pretreatment step and the proliferation step may be repeated any number of times.

Thus, the pretreatment step according to the present invention can improve the proliferation rate in the proliferation step and therefore the concentration of microorganisms can also be increased. The greater the aggregation properties of microorganisms, the more effective the pretreatment step. More specifically, microorganisms present on the surface of aggregates in contact with a medium are accessible to many nutrients; in the case of photosynthetic microorganisms, the microorganisms can obtain a sufficient amount of light. Further, it is considered that since there are also spaces for proliferation, microorganisms can grow vigorously. On the other hand, nutrients are not sufficiently diffused to microorganisms on the inside of aggregates because many microorganisms exist before reaching the medium. Or due to the consumption of nutrients by microorganisms closer to the medium, the microorganisms on the inside of aggregates have less opportunity to get nutrients and thus have difficulty in receiving nutrients. Moreover, there is little space for proliferation and little light reaches photosynthetic microorganisms. Thus, there is a problem that proliferation is suppressed inside of aggregates.

So by carrying out the pretreatment step according to the present invention, aggregates can be disaggregated into smaller aggregates or individual microorganisms while preventing disruption of microorganisms, and therefore the above problem can be avoided. As a result, proliferation rates of microorganisms can be improved.

Use of the pretreatment step according to the present invention can also increase the concentration of microalgae which produce biomass material, in a culture solution. Microalgae which produce biomass material, for example, Botryococcus Braunii which is known to accumulate oil equivalent to heavy oil inside and/or outside the microbial bodies, have an extremely low proliferation rate of a few days to a few weeks. For this reason, despite receiving much attention worldwide, commercial scale production has not been attained.

So far studies have been made to improve their proliferation rate by optimizing the amount of light, optimizing the interval of the irradiation of light or precisely investigating the composition of media, but the improvement is still unsatisfactory at present. Botryococcus Braunii is known to form aggregates with extracellular matrix and it is considered that the facts described above cause low proliferation rates.

So by employing the pretreatment step according to the present invention, aggregates can be disaggregated and separated into pieces while preventing disruption of microorganisms, and therefore the aforementioned problem caused by aggregation properties can be eliminated. As a result, more individual microorganisms can be involved in proliferation. This can remarkably improve the proliferation rate of Botryococcus Braunii and thus may enable commercial scale production.

### [Intended Treatment Step (Purification Treatment of Microorganisms)]

The purification treatment in the present invention means a technique for obtaining a complex of a single type of microorganisms from a complex of a plurality of types of microorganisms. Generally the purification treatment includes the following steps. First, complexes of microorganisms collected from nature are developed on agar gel, and cultured and proliferated, and the resulting colonies are then collected. Then whether the colonies collected are composed of a single microbe or not is observed. At this stage, when the resulting colonies are composed of a plurality of types of microorganisms, the colonies are developed on an agar medium again. The purification steps are repeated until a single microbe is obtained.

In the case of microorganisms which do not form aggregates, the microorganisms can be purified by forming good colonies by appropriately reducing the concentration of microorganisms as in conventional methods.

However, most of the microorganisms collected from nature which form aggregates are a mixture of microorganisms containing numerous types of microorganisms in the aggregates. And in such cases, when microorganisms are purified as in conventional methods, it is highly likely that several types of microorganisms are mixed in colonies even if colonies are formed. Thus it has been impossible to purify microorganisms unless the purification steps are repeated many times.

However, when the pretreatment step according to the present invention is employed, aggregates of microorganisms are disintegrated into individual microorganisms in the pretreatment step, and colonies composed of a single type of microorganisms can be formed. As a result, colonies composed of a single type of microorganisms can be obtained more easily with fewer purification steps.

For example, microalgae having aggregation properties such as Botryococcus Braunii form aggregates by being combined with each other via extracellular matrix. It is said that bacteria or the like enter into the extracellular matrix and this is the reason why purification of such microalgae is very difficult.

However, by carrying out the pretreatment step according to the present invention, aggregates can be disaggregated and separated into a state close to single bodies while preventing disruption of microalgae. As a result, microalgae and bacteria are apart from each other. This can facilitate the purification of microalgae. Upon the purification treatment, a centrifugation treatment or a filtration treatment with a porous film may also be performed after the pretreatment step of a sample solution. For example, since microalgae and bacteria have a different size, it is even more preferred that they are separated by using a porous film or by centrifugation utilizing the difference in specific gravities after the pretreatment step. This makes the purification treatment much easier.

Also, to kill coexisting bacteria, purification may be carried out using an antibacterial agent. However, the antibacterial agent hardly permeates bacteria present in extracellular matrix, and thus has a very limited effect.

Even in such a case, extracellular matrix is separated into pieces or fragmented further by carrying out the pretreatment step according to the present invention. This improves the permeability of antibacterial agents and thus can make the antibacterial agents more effective.

Also, when the above techniques are used, purification of a group of microorganisms to be purified will proceed to some extent upon purification. Therefore, considering the advantage of the pretreatment step according to the present invention (i.e., the presence of individual microorganisms) at the same time, the number of purification steps can be reduced and the time for the purification treatment can be significantly reduced. Accordingly, use of the method of the present invention makes the purification step of microorganisms easier and can significantly reduce the time for purification.

### EXAMPLES

The present invention will be described in more detail with reference to Examples below, but the present invention is not limited thereto.

### Example A

In Example A, the disaggregation effect on aggregates of microorganisms and the condition of disruption of microorganisms are each compared in the pretreatment step according to the present invention of subjecting a closed container in which a microorganism-containing sample solution is packed to high speed oscillating motion and in a conventional ultrasonic treatment step of irradiating a closed container in which a microorganism-containing sample solution is packed with ultrasonic waves. At the same time, the condition of disruption of microorganisms when a closed container in which a sample solution was packed was subjected to high speed oscillating motion with beads added thereto was examined.

### (Preparation of Sample)

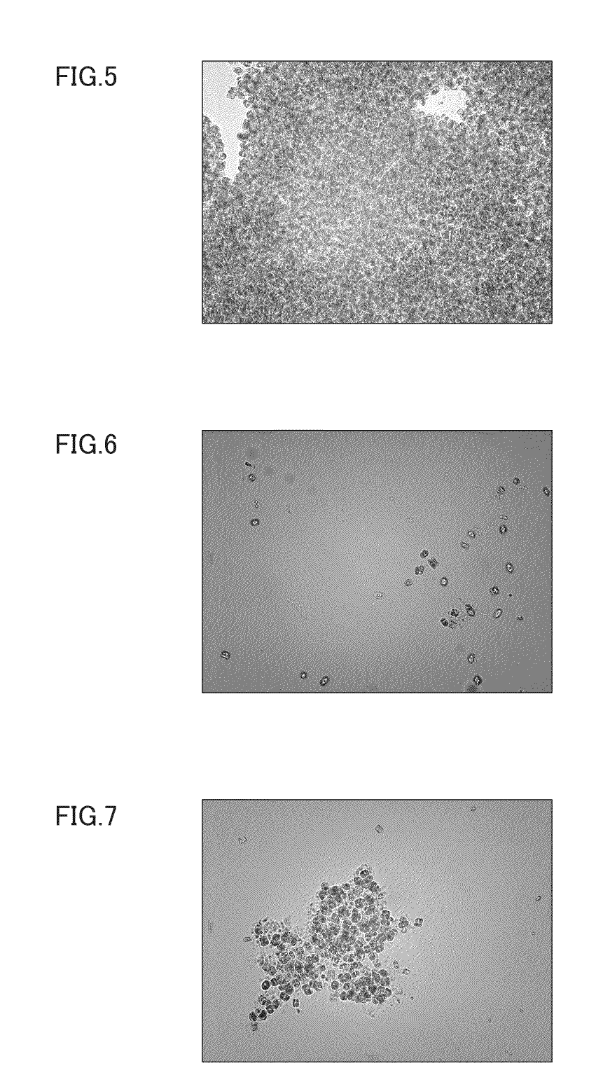

A 500 mL conical flask was charged with 200 mL of a sterilized IMK medium and microalgae (one of diatoms) collected from natural seawater were inoculated on the medium. Then the microalgae were cultured using a shaking incubator (Incubator Shaker Model RGS-20RL) at an illuminance of 2000 LUX at 20° C. and a shaking speed of 100 rpm. FIG. 5 shows the result of the observation of the resulting sample solution containing microalgae (diatoms) by a microscope (magnification: 4 times). Since the diatom used in this Example has high aggregation properties and forms aggregates as shown in FIG. 5, it was impossible to count individual (single) microalgae even in the observation by the microscope.

The aggregates of microalgae shown in FIG. 5 were treated as a test sample in the pretreatment step according to the present invention (Example 1) and in a conventional ultrasonic treatment (Comparative Example 1) as follows.

### Example 1

0.5 mL of the sample solution of microalgae cultured as described above was collected and put in a 2 mL closed container 22 with a lid 22A shown in FIG. 3. The closed container 22 was set on the pretreatment apparatus 10. The volume ratio of the sample solution to the inner volume of the closed container 22 is 25% by volume. Beads Cell Disrupter MS-100 made by TOMY SEIKO CO., LTD. which is for high speed oscillating motion in a vertical direction and in a figure of eight was used as the pretreatment apparatus 10. No beads were added to the closed container 22. Then the pretreatment step was performed at an oscillating number of 5500 rpm for 20 seconds. The result of the observation of the microalgae after the pretreatment step by a microscope (magnification: 10 times) is shown in FIG. 6.

As is apparent from FIG. 6, aggregates of microalgae were disaggregated into single microalgae and the individual microalgae could be easily counted with a microscope. Further, whether the microalgae after the pretreatment step were disrupted or not was observed using a microscope (magnification: 10 times). The collected sample (10 μL) put on a slide glass was observed, and no disruption of the microalgae was found within the area of the cover glass (22 mm×22 mm)

### Comparative Example 1

0.5 mL of the sample solution cultured as described above was collected and put in a 2 mL closed container 22 with a lid 22A shown in FIG. 3. The closed container 22 was set on Ultrasonic Cleaner USD-4R (made by AS ONE Corporation). Then after applying ultrasonic waves to the sample solution at 28 kHz for 5 minutes, microalgae were observed with a microscope.

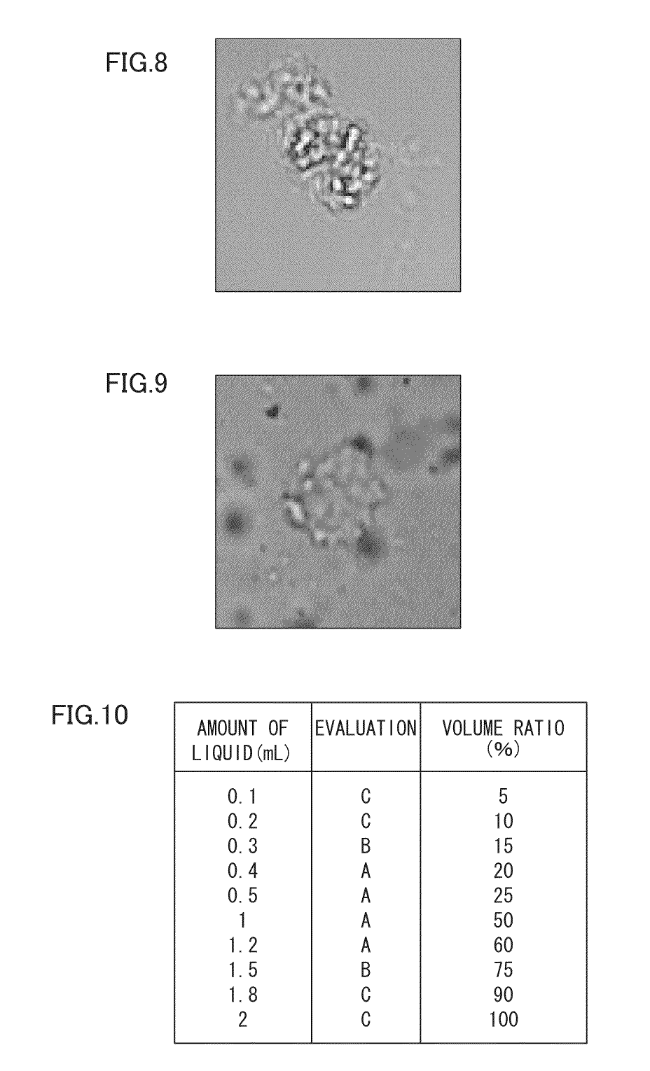

The results are shown in FIG. 7 and FIG. 8. FIG. 7 (magnification: 10 times) shows that the condition of dispersion of aggregates of microalgae was similar to that before applying ultrasonic waves and the level of dispersion by applying ultrasonic waves was obviously poorer than that in Example 1 (FIG. 6). Further, whether the microalgae after the ultrasonic treatment were disrupted or not was observed using a microscope (magnification: 40 times), and as a result, some cells were broken. As described above, the ultrasonic treatment has resulted in unsuccessful dispersion and disruption of some cells (FIG. 8).

As is apparent from the comparison between Example 1 and Comparative Example 1, while the disaggregation efficiency was low and some cells were broken in the conventional ultrasonic treatment, a high disaggregation efficiency was shown and the disruption of microalgae was effectively prevented in Example 1 in which a predetermined amount of a sample solution was packed in the closed container 22 and the closed container 22 was subjected to high speed oscillating motion in the pretreatment apparatus 10.

### Comparative Example 2

0.5 mL of the sample solution cultured as described above was collected and put in a 2 mL closed container 22 with a lid 22A, and 20 zirconia beads having a diameter of 2 mm were also put in the closed container 22. The closed container 22 was set on the pretreatment apparatus 10 as in Example 1 and subjected to high speed oscillating motion at 5500 rpm for 20 seconds as in Example 1.

The results are shown in FIG. 9 (magnification: 10 times). As is apparent from FIG. 9, when zirconia beads were put in the closed container 22, cells of microalgae were disrupted and it was impossible to accurately count the number of microalgae.

As is apparent from the test results of Example 1 and Comparative Example 2, basically putting beads such as zirconia beads in the closed container 22 in the pretreatment step according to the present invention is not preferred to achieve the effect of disaggregating aggregates while preventing disruption of microorganisms. Therefore it is preferred that the high speed oscillating motion is carried out by changing the combination of the oscillating number and the oscillating time for the closed container 22. However, since there may be a case where aggregation properties of microorganisms are too great to achieve a sufficient disaggregation effect, beads may be used after determining conditions (the diameter and the number of beads) which do not cause disruption of microorganisms by a preliminary experiment or the like. In short, aggregates are to be efficiently disaggregated while preventing disruption of microorganisms.

### Example B

In Example B, preferred ranges of the volume ratio of a sample solution to the inner volume of the closed container 22 in the pretreatment step were examined.

Ten 2 mL closed containers 22 with a lid 22A were prepared and a sample solution of microalgae cultured as in Example A was packed in the respective containers in a volume of 0.1 mL (volume ratio 5%), 0.2 mL (volume ratio 10%), 0.3 mL (volume ratio 15%), 0.4 mL (volume ratio 20%), 0.5 mL (volume ratio 25%), 1.0 mL (volume ratio 50%), 1.2 mL (volume ratio 60%), 1.5 mL (volume ratio 75%), 1.8 mL (volume ratio 90%) and 2.0 mL (volume ratio 100%). No beads were used. The 10 closed containers 22 were set on the same pretreatment apparatus 10 as used in Example A and subjected to the pretreatment step at 5500 rpm for 20 seconds.

The results are shown in the table in FIG. 10. The disaggregation effect was evaluated and rated as follows.

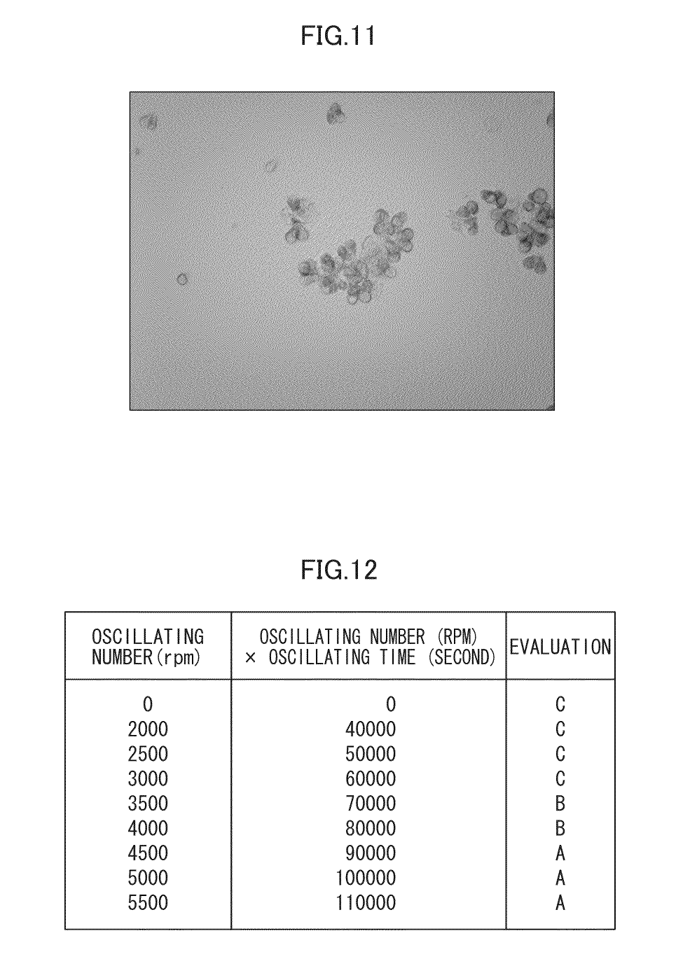

A: Disaggregated to the same extent as in FIG. 6; the number of microalgae can be easily counted with a microscope.
B: Aggregates remain but are small as shown in FIG. 11; the number of microalgae can be counted.
C: Aggregates are large as shown in FIG. 5 and FIG. 7; counting of the number of microalgae is difficult.

As is apparent from the results in the table in FIG. 10, those in which the packing amount in the closed container 22 was 0.1 mL (volume ratio 5%) and 0.2 mL (volume ratio 10%) were evaluated as C, showing a low disaggregation effect, and the counting of microalgae was difficult. One in which the packing amount was increased to 0.3 mL (volume ratio 15%) was evaluated as B and those in which the packing amount was 0.4 mL (volume ratio 20%) to 1.2 mL (volume ratio 60%) were evaluated as A. One in which the packing amount was further increased to 1.5 mL (volume ratio 75%) was evaluated as B and those in which the packing amount was 1.8 mL (volume ratio 90%) or more were evaluated as C.

As described above, aggregates could not be disaggregated well when packing the amount of the sample solution packed in the closed container 22 was too large or too small. From this, it is considered that the head space in the closed container 22 upon the high speed oscillating motion greatly affects the disaggregation efficiency for aggregates. Also, the reason why the dispersibility is poor when the packing amount is too small seems to be because the weight of the sample solution is too low and so the collision force against the inner wall of the closed container upon high speed oscillating motion is small, and therefore energy needed to disperse aggregates cannot be fully achieved.

When considering preferred amounts of a sample solution to be packed in the closed container 22 in terms of the volume ratio of the sample solution to the inner volume of the closed container 22, the test results in FIG. 10 show that the volume ratio is preferably in the range of 15% by volume to 75% by volume and more preferably 20% by volume to 60% by volume.

### Example C

In Example C, preferred oscillating numbers and oscillating times of the high speed oscillating motion in the pretreatment step were examined.

Nine 2 mL closed containers 22 with a lid 22A were prepared and 0.5 mL of a sample solution of microalgae cultured as in Example A was packed in the containers. No beads were used. The nine closed containers 22 were set on the same pretreatment apparatus 10 as used in Example A and subjected to the pretreatment step per test lot. More specifically, the closed containers 22 were each subjected to high speed oscillating motion at an oscillating number of 2,000 rpm, 2,500 rpm, 3,000 rpm, 3,500 rpm, 4,000 rpm, 4,500 rpm, 5,000 rpm or 5,500 rpm for an oscillating time of 20 seconds.

The results are shown in the table in FIG. 12. The disaggregation effect was evaluated and rated as in Example B.

As FIG. 12 shows, those in which the oscillating number was 0 (without high speed oscillating motion) to 3,000 rpm (oscillating number×oscillating time being 0 to 60,000) were evaluated as C, while those in which the oscillating number was 3,500 to 4,000 rpm (oscillating number×oscillating time being 70,000 to 80,000) were improved and evaluated as B and those in which the oscillating number was 4,500 to 5,500 rpm (oscillating number×oscillating time being 90,000 to 110,000) were evaluated as A. The results show that when the oscillating time is as relatively short as 20 seconds, the oscillating number should be 3,500 rpm or more, and is more preferably 4,500 rpm or more.

Given this, of the oscillating numbers ranging from 0 to 5,500 rpm employed in the test, two cases of 3,500 rpm evaluated as B and 5,500 rpm evaluated as A were tested with changing the oscillating time from 5 seconds to 60 seconds.

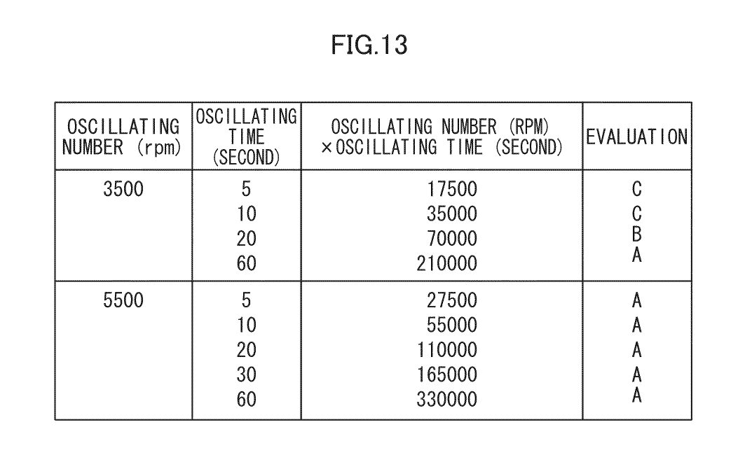

The results are shown in the table in FIG. 13. As FIG. 13 shows, at an oscillating number of 3,500 rpm, those in which the oscillating time was up to 10 seconds (oscillating number×oscillating time being 35,000) were evaluated as C, while one in which the oscillating time was 20 seconds (oscillating number×oscillating time being 70,000) was evaluated as B and one in which the oscillating time was 60 seconds (oscillating number×oscillating time being 210,000) was evaluated as A. On the other hand, at an oscillating number of 5,500 rpm, even one in which the oscillating time was 5 seconds (oscillating number×oscillating time being 27,500) was evaluated as A and those in which the oscillating time was up to 60 seconds (oscillating number×oscillating time being 330,000) were evaluated as A.

The test results in the table in FIG. 12 and the table in FIG. 13 show that it is preferred that the high speed oscillating motion for microalgae is carried out within an oscillating time of 60 seconds in consideration of the influence on the disruption. For that purpose, the oscillating number is preferably 3500 rpm or more, more preferably 5,500 rpm to 10,000 rpm. The upper limit of the oscillating number is 10,000 rpm because when the oscillating number is more than 10,000 rpm, a greater amount of heat is generated in the pretreatment apparatus 10, and an increase in temperature caused by the heat generation is undesirable for the activity of common microorganisms.

Accordingly, to sum up the above results, it is preferred that in the high speed oscillating motion for the closed container 22, the oscillating number (rpm)×oscillating time (second) ranges from 70,000 to 330,000.

### Example D

In Example D, the advantageous effect of the method of treating microorganisms of the present invention was examined. More specifically, the proliferation effect when the pretreatment step is included in a proliferation treatment which is an embodiment of the intended treatment steps was examined.

1 mL of a sample solution of microalgae cultured as in Example A was packed in a 2 mL closed container 22 with a lid (volume ratio: 50% by volume) and the pretreatment step was performed at 5,500 rpm for 20 seconds (oscillating number×oscillating time being 110,000) to disperse the microalgae. The sample solution after the pretreatment step was inoculated on 200 mL of a sterilized IMK medium in a 500 mL conical flask, and was cultured using a shaking incubator (RGS-20RL) at an illuminance of 2,000 LUX at 20° C. and a shaking speed of 100 rpm. As a result, brown aggregates of microalgae were formed in the medium.

The brown aggregates prepared as described above was collected as a test sample and divided in half, and 1 mL of one of them (test sample 1) was packed in a 2 mL closed container 22 with a lid. The aggregates were separated into pieces by carrying out the pretreatment step again at 5,500 rpm for 20 seconds (oscillating number×oscillating time being 110,000). Then the test sample 1 after the pretreatment step was inoculated on 200 mL of a sterilized IMK medium in a 500 mL conical flask.

At the same time, the other half of the aggregates (test sample 2) was inoculated on 200 mL of a sterilized IMK medium in a 500 mL conical flask without the pretreatment. The amount of inoculation of the test sample 1 and test sample 2 was both 2.5×10⁴ pieces/mL in terms of the concentration of the algal bodies. The amount of inoculation was identified by counting the number of algal bodies in samples prepared by pretreating the test samples 1 and 2 in the pretreatment apparatus 10 at 5,500 rpm for 20 seconds (oscillating number x oscillating time being 110,000) using a hemocytometer.

Next, the conical flasks of the test sample 1 and the test sample 2 were each set on a shaking incubator (RGS-20RL) and the samples were cultured at an illuminance of 2,000 LUX at 20° C. and a shaking speed of 100 rpm for 17 days. The culture solution was taken out from each of the conical flasks of the test sample 1 and the test sample 2 after 3 days, 5 days, 10 days and 17 days of the cultivation, and 0.5 mL of the culture solution was put in a 2 mL closed container 22 with a lid. After setting the closed container 22 on the pretreatment apparatus 10, the pretreatment was carried out at 5,500 rpm for 20 seconds (oscillating number x oscillating time being 110,000), and then the number of microalgae was counted using a hemocytometer.

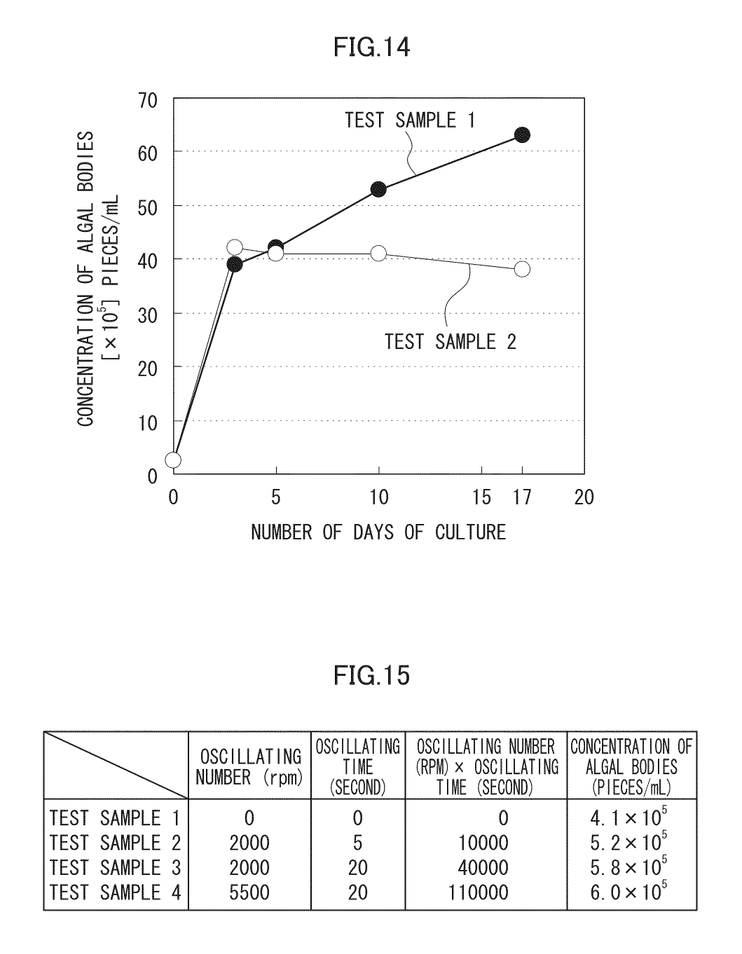

The relationship between the number of days of culture and the concentration of algal bodies is shown in the graph in FIG. 14.

As is apparent from the results in FIG. 14, until the 5th day of culture, the concentration of algal bodies was almost the same in the test sample 1 for which the culturing step was carried out after the pretreatment step and the test sample 2 for which the culturing step was carried out without the pretreatment step.

However, after the 5th day of culture, while the curve of the concentration of algal bodies of the test sample 1 slopes upward, the curve of the concentration of algal bodies of the test sample 2 is flat. More specifically, while the concentration of algal bodies of the test sample 1 increased to 63×10⁵ pieces/mL, the concentration of algal bodies of the test sample 2 was 39×10⁵ pieces/mL on the 17th day of culture.

### Example E

In Example E, the change in culture rates when the conditions of the pretreatment step (oscillating number and oscillating time) in Example D were changed was examined.

A test sample containing aggregates of microalgae was prepared by the same procedure as in Example D. Next, four 2 mL closed containers 22 with a lid were prepared and the test sample was divided into four portions (test samples 1, 2, 3, 4). 0.5 mL of the test samples 1 to 4 was packed in the respective containers.

The test samples 1 to 4 were subjected to the pretreatment step under the following conditions.
Test sample 1: oscillating number: 0 rpm, oscillating time: 0 second (without pretreatment step)
Test sample 2: oscillating number: 2,000 rpm, oscillating time 5 seconds (oscillating number×oscillating time being 10,000)
Test sample 3: oscillating number: 2,000 rpm, oscillating time 20 seconds (oscillating number×oscillating time being 40,000)
Test sample 4: oscillating number: 5,500 rpm, oscillating time 20 seconds (oscillating number×oscillating time being 110,000)

The test samples 1 to 4 prepared as described above were each inoculated on 25 mL of a sterilized IMK medium in a 100 mL conical flask, and cultured using a shaking incubator (RGS-20RL) at an illuminance of 2,000 LUX at 20° C. and a shaking speed of 100 rpm.

Then culture was terminated on the 10th day from the start of the culture, and 0.5 mL of the samples (test samples 1 to 4) was collected from the respective conical flasks. The samples collected were each packed in a 2 mL closed container 22 with a lid and the closed containers were set on the pretreatment apparatus 10. The samples were disaggregated by carrying out the pretreatment step at 5500 rpm for 20 seconds, and then the number of algal bodies was counted using a hemocytometer.

The results are shown in the table in FIG. 15. The concentration of algal bodies in the 100 mL conical flasks was 1.6×10⁵ pieces/mL at the start of the culture.

As is apparent from the results in FIG. 15, the test sample 1 for which the culturing treatment was carried out without the pretreatment step had a concentration of microalgae of 4.1×10⁵ pieces/mL.

On the other hand, the test sample 2 for which the pretreatment step was carried out at 2,000 rpm for 5 seconds and then the culturing treatment was carried out had a concentration of microalgae of 5.2×10⁵ pieces/mL, and the test sample 3 for which the pretreatment step was carried out at 2,000 rpm for 20 seconds had a concentration of microalgae of 5.8×10⁵ pieces/mL. Further, the test sample 4 for which the pretreatment step was carried out at 5,500 rpm for 20 seconds and then the culturing treatment was carried out had a concentration of microalgae of 6.0×10⁵ pieces/mL.

As the comparison between the test samples 2 and 3 shows, when the oscillating numbers are the same, the longer the oscillating time, in other words, the greater the degree of dispersion of aggregates of microorganisms, the more the culture rate in the culturing treatment improves. Also, as the comparison between the test samples 3 and 4 shows, when the oscillating times are the same, the larger the oscillating number, in other words, the greater the degree of dispersion of aggregates of microorganisms, the more the culture rate in the culturing treatment improves.

The results of Examples D and E described above show that the culture rate can be improved by carrying out a pretreatment step of packing a predetermined amount of a sample solution containing microorganisms in a closed container 22 and subjecting the closed container 22 to high speed oscillating motion before carrying out a culturing treatment which is an embodiment of the intended treatment steps. This is considered to be because the inclusion of the pretreatment step in a proliferation treatment of microorganisms enables efficient dispersion of aggregates of microorganisms while preventing disruption of microorganisms, and thus individual microorganisms have an easy access to components necessary for proliferation, such as nutrient sources, and can take in the components sufficiently. As a result, proliferation rates can be significantly improved compared to those in conventional methods even for microorganisms having a low proliferation rate.

In consideration of the relationship between the concentration of algal bodies which indicates the culture rate and the (oscillating number×oscillating time), the pretreatment step is effective when the oscillating number×oscillating time is 10000 or more, showing a good result even with a value lower than the lower limit of oscillating number×oscillating time of 70000 in the test for examining the disaggregation efficiency in Example C (FIG. 12, FIG. 13).

What is claimed is:

1. A method of treating microorganisms in which any one of the intended treatments of a treatment of counting the number of microorganisms, a proliferation treatment of microorganisms and a purification treatment of microorganisms is carried out, the method comprising:
before carrying out the intended treatment step, a pretreatment step of packing a predetermined amount of a sample solution containing the microorganisms in a closed container and subjecting the closed container to high speed oscillating motion.

2. The method of treating microorganisms according to claim 1, wherein the predetermined amount is such that a volume ratio of the sample solution to the inner volume of the closed container is 15 to 75% by volume.

3. The method of treating microorganisms according to claim 1, wherein, in the high speed oscillating motion, the closed container is oscillated so that a value of oscillating number (rpm)×oscillating time (second) ranges 10,000 to 3,000,000.

4. The method of treating microorganisms according to claim 2, wherein, in the high speed oscillating motion, the closed container is oscillated so that a value of oscillating number (rpm)×oscillating time (second) ranges 10,000 to 3,000,000.

5. The method of treating microorganisms according to claim 1, wherein an oscillating pattern for the closed container is at least one pattern in a vertical direction, a horizontal direction or a back and forth direction.

6. The method of treating microorganisms according to claim 4, wherein an oscillating pattern for the closed container is at least one pattern in a vertical direction, a horizontal direction or a back and forth direction.

7. The method of treating microorganisms according to claim 1, wherein, in the pretreatment step, an antifoaming agent is added to the closed container in which the sample solution is packed.

8. The method of treating microorganisms according to claim 6, wherein, in the pretreatment step, an antifoaming agent is added to the closed container in which the sample solution is packed.

9. The method of treating microorganisms according to claim 1, wherein the pretreatment step is carried out in several stages.

10. The method of treating microorganisms according to claim 8, wherein the pretreatment step is carried out in several stages.

11. The method of treating microorganisms according to claim 1, wherein the microorganisms have aggregation properties.

12. The method of treating microorganisms according to claim 10, wherein the microorganisms have aggregation properties.

13. The method of treating microorganisms according to claim 11, wherein the microorganisms having aggregation properties are microalgae.

14. The method of treating microorganisms according to claim 12, wherein the microorganisms having aggregation properties are microalgae.

15. The method of treating microorganisms according to claim 1, wherein the microorganisms are a complex including a plurality of types of microorganisms.

16. The method of treating microorganisms according to claim 10, wherein the microorganisms are a complex including a plurality of types of microorganisms.

17. The method of treating microorganisms according to claim 1, wherein the microorganisms produce biomass material.

18. The method of treating microorganisms according to claim 16, wherein the microorganisms produce biomass material.

19. The method of treating microorganisms according to claim 17, wherein the microorganisms which produce the biomass material are of the genus Botryococcus.

20. The method of treating microorganisms according to claim 18, wherein the microorganisms which produce the biomass material are of the genus Botryococcus.

21. The method of treating microorganisms according to claim 1, wherein the pretreatment step and the intended treatment step are repeated several times when the intended treatment step is the proliferation treatment of microorganisms.

22. The method of treating microorganisms according to claim 20, wherein the pretreatment step and the intended treatment step are repeated several times when the intended treatment step is the proliferation treatment of microorganisms.
